(19) United States
(12) Patent Application Publication
(10) Pub. No.: US 2022/0347256 A1
(43) Pub. Date: Nov. 3, 2022

(54) COMPOSITION FOR PROMOTING HAIR GROWTH, AND ALLEVIATING AND TREATING HAIR LOSS INCLUDING SUBSTANCE P

(71) Applicant: Biosolution Co., Ltd., Seoul (KR)

(72) Inventors: Junho KIM, Seoul (KR); Da Jung KIM, Seoul (KR); Ga Eun YOU, Seoul (KR); Jungsun LEE, Seoul (KR); Song Sun CHANG, Seoul (KR)

(21) Appl. No.: 16/619,935

(22) PCT Filed: Oct. 29, 2019
(86) PCT No.: PCT/KR2019/014378

§ 371 Date: Dec. 5, 2019

(30) Foreign Application Priority Data
May 22, 2019 (KR) 10-2019-0060060

(51) Int. Cl. A61K 38/08; A61K 47/02; A61K 47/26; A61K 47/38; A61P 17/14

(52) U.S. Cl. CPC A61K 38/08; A61K 47/02; A61K 47/26; A61K 47/38; A61P 17/14

(57) ABSTRACT

The present invention relates to a composition for promoting hair growth, and alleviating and treating hair loss, comprising substance P (SP) as an active ingredient. More specifically, the present invention relates to a pharmaceutical composition, which comprises substance P, for promoting hair growth, and preventing or treating hair loss and a cosmetic composition, which comprises substance P, for promoting hair or alleviating hair loss by increasing the activity of hair follicle cells, maintaining the growth phase of hair follicles, and inhibiting the progression into the regression phase. The composition of the present invention, which comprises substance P, an antioxidant, a surfactant, and a thickening agent, can be used for application to medicines, cosmetics, etc., for promoting hair growth, and alleviating and treating hair loss.

### CROSS REFERENCE TO RELATED APPLICATIONS

This application is a National Stage Application filed under 35 U.S.C. § 371 and claims priority to International Application No. PCT/KR2019/014378, filed Oct. 29, 2019, which application claims priority to Korean Patent Application No. 10-2019-0060060, filed May 22, 2019, the disclosures of which are incorporated herein by reference.

### TECHNICAL FIELD

The present invention relates to a composition for promoting hair growth, and alleviating and treating hair loss, including substance P (SP) as an active ingredient. More specifically, the present invention relates to a pharmaceutical composition, which includes substance P, for promoting hair growth, and preventing or treating hair loss and a cosmetic composition, which includes substance P, for promoting hair or alleviating hair loss by increasing the activity of hair follicles, maintaining the growth phase of hair follicles, and inhibiting the progression into the regression phase.

### BACKGROUND

Currently, the interest in hair loss inhibition is gradually increasing. The world's hair loss population is steadily increasing, and minoxidil is widely used as a medicine to induce hair growth effects. However, the principle of minoxidil for inducing hair growth lies in vasodilation, and thus, it induces the dilation of arteries but maintains the vein diameter, which leads to an overall difference in pressure, and additionally, it has a disadvantage in that it is difficult to use minoxidil in combination with medication for high blood pressure. Further, the major side effect of minoxidil includes unintentional hirsutism in female patients since minoxidil induces hair growth in a large area rather than a topical area. As a result, more than half of hair loss patients prefer to use hair loss-alleviating product so as to avoid the side effects of currently-available medications.

Hair loss-alleviating products are commercially available, which have fewer relevant side effects and lower risk for women and patients taking medication for high blood pressure in combination. Typically, hair components, such as biotin, nicotinic acid amide, pyrimidine zinc solution-based or plant-based complexes, etc. are supplied to alleviate hair loss. To date, medications based on supplying nutrients are significantly less effective than minoxidil.

Substance P (SP) is a peptide composed of 11 amino acids and is a neurotransmitter scattered in the nervous tissue. It has long been known to be involved in the activity of various cells in the body, such as nerve cells, blood cells, epithelial cells, etc., and is recently known to play a role in anti-inflammation, angiogenesis, etc. and thus has a wide range of applications. In particular, the result of increasing the activity of blood vessels, epithelial cells, and fibroblasts is closely related to the induction of hair follicle growth phase. Additionally, studies on inducing mesenchymal stem cells to bone differentiation have discovered a mechanism by which substance P activates the TCF family, a mediator that delivers Wnt/β-catenin signaling to the nucleus. Activating such mediator of Wnt/β-catenin signaling is an important factor for the inhibition of androgenetic alopecia. In androgenetic alopecia, androgen expresses DKK-1 in dermal papilla cells and induces the degradation of β-catenin, thereby inhibiting factors involved in the growth phase and activating factors involved in the regression phase, resulting in degeneration of hair cells. If the substance P in hair maintains its characteristics in helping the growth of dermal papilla cells and activating the TCF-family, it is expected to function in maintaining the growth phase of hair even in hair loss conditions.

From the same perspective, several literatures have studied the relationship between substance P and induction of the growth phase of hair follicles, and certain studies have confirmed the result that the substance P helps the proliferation of hair follicles in vitro. However, due to the nature of substance P that it is easily degraded in the body, its effect on hair growth in hair loss conditions is unclear, and in particular, it is difficult to reach conclusions since the solvents and concentrations of substance P differ from literature to literature.

Since the substance P is a peptide that is easily degraded after the synthesis thereof, it is difficult to apply to harsh environments such as bioinjection. In the present invention, based on the studies of developing a formulation that improves the stability of substance P, a composition with improved efficacy was implemented by preventing the degradation of substance P. Through this finding, the effect of inducing the growth phase and inhibiting the regression phase of hair follicles was confirmed by improving the stability of the synthesized substance P in vitro and injecting the same into the mice body.

### DISCLOSURE

### Technical Problem

Under the circumstances, the present inventors have made extensive efforts to increase the stability of substance P, and as a result, they have found that a composition including substance P, an antioxidant, a surfactant, and a thickening agent shows a more superior effect of promoting hair growth, and alleviating and treating hair loss by increasing the activity of hair follicles, maintaining the growth phase of hair follicles and inhibiting the progression into the regression phase, as compared to the substance P itself, thereby completing the present invention.

### Technical Solution

One object of the present invention is to provide a pharmaceutical composition, which includes substance P, for promoting hair growth, and preventing or treating hair loss.

Another object of the present invention is to provide a cosmetic composition, which includes substance P, for promoting hair growth or alleviating hair loss.

### Advantageous Effects

The composition of the present invention, which includes substance P, an antioxidant, a surfactant, and a thickening agent, can be used for application to medicines, cosmetics, etc., for promoting hair growth, and alleviating and treating hair loss.

### BRIEF DESCRIPTION OF DRAWINGS

FIG. 1 is a graph showing the effect of the composition including the substance P in the proliferation of dermal papilla cells.

FIG. 2 shows the PCR results of maintaining the characteristics of the growth phase of the dermal papilla cells proliferated by the composition including the substance P.

FIG. 3 is a graph showing the composition including the substance P in the proliferation of germinal matrix cells.

FIG. 4 are images confirming the hair growth effect of the composition including the substance P in C3H mice.

FIG. 5 is a graph confirming the effect of inducing hair growth on the back of C3H mice by the composition including the substance P through hair shaft area.

FIG. 6 is a graph confirming the effect of inducing hair growth on the back of C3H mice by the composition including the substance P through hair length.

FIG. 7 shows images confirming the effect of inducing hair growth on the back of C3H mice by the composition including the substance P through H&E staining of skin tissues.

FIG. 8 is a graph confirming the effect of inducing hair growth on the back of C3H mice by the composition including the substance P through hair skin thickness.

FIG. 9 is a graph confirming the effect of inducing hair growth on the back of C3H mice by the composition including the substance P through the number of hair follicles in the skin tissues

FIG. 10 shows images confirming the degree of maintenance of skin in the growth phase for each treatment group in C3H mice in which the regression phase was induced by dexamethasone.

FIG. 11 is a graph showing the area of the skin, in which the growth phase was maintained, relative to the total skin area for each treatment material in C3H mice in which the regression phase was induced by dexamethasone.

FIG. 12 shows images confirming the shape of individual hair follicles in the skin tissue for each treatment group in C3H mice in which the regression phase was induced by dexamethasone.

FIG. 13 is a graph showing the thickness of the dermis/subcutaneous/whole layers in the skin tissue for each treatment group in C3H mice in which the regression phase was induced by dexamethasone.

FIG. 14 shows images confirming the cross-sectional shape and depth of individual hair follicles contained in the skin tissue for each treatment group in C3H mice in which the regression phase was induced by dexamethasone.

FIG. 15 is a graph analyzing the number of hair follicles in the dermis/subcutaneous/whole layers in the skin tissue for each treatment group in C3H mice in which the regression phase was induced by dexamethasone.

### DETAILED DESCRIPTION OF PREFERRED EMBODIMENTS

In order to achieve the objects above, an aspect of the present invention provides a pharmaceutical composition, which includes substance P, an antioxidant, a surfactant, and a thickening agent, for promoting hair growth, and preventing or treating hair loss.

Additionally, another aspect of the present invention provides the use of a composition, which includes substance P, an antioxidant, a surfactant, and a thickening agent, for promoting hair growth, and preventing or treating hair loss.

The present invention relates to a novel pharmaceutical composition for improving the insignificant efficacy of the existing substance P, due to instability, in promoting hair growth, and preventing or treating hair loss. The present inventors have found the components and contents of the composition which can exhibit an optimal effect of promoting hair growth, and preventing or treating hair loss upon application of substance P.

Specifically, the present invention provides a pharmaceutical composition, which includes substance P, sodium thiosulfate, polysorbate 80, and hydroxyethyl cellulose, for promoting hair growth, and preventing or treating hair loss.

The present inventors have found that the composition including the substance P shows an effect of promoting hair growth, and preventing or treating hair loss by promoting the proliferation of dermal papilla cells and germinal matrix cells, and growth of hair follicles cells, and controlling the growth phase of hair, thereby completing the present invention. In the composition of the present invention, the substance P refers to a neuropeptide consisting of the amino acid “Arg-Pro-Lys-Pro-Gln-Gln-Phe-Phe-Gly-Leu-Met-NH₂” of SEQ ID NO: 1.

The concentration of substance P contained in the composition of the present invention may be 6 μg/mL to 10 μg/mL, specifically, 7 μg/mL to 10 μg/mL, 8 μg/mL to 10 μg/mL, or 9 μg/mL to 10 μg/mL.

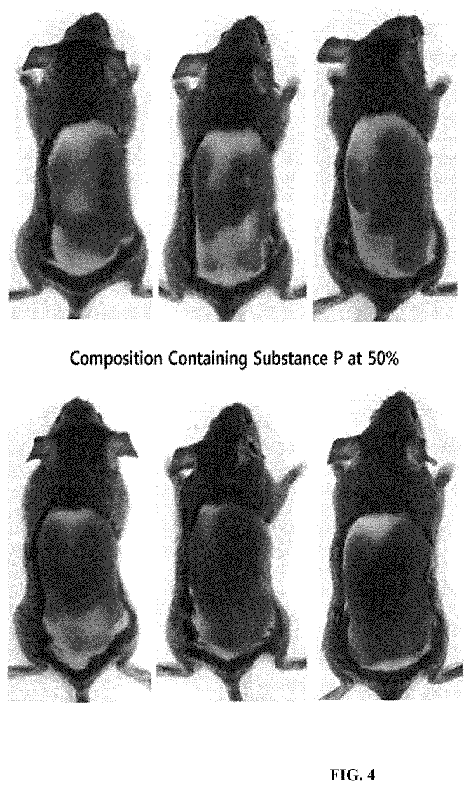

In one embodiment of the present invention, the composition including the substance P at a concentration of 6 μg/mL to 10 μg/mL showed the most superior effect of promoting hair growth, and preventing or treating hair loss (FIG. 4).

Therefore, in the pharmaceutical composition of the present invention, when the substance P is contained at a concentration of 6 μg/mL to 10 μg/mL, it was confirmed that the pharmaceutical composition of the present invention has an excellent effect of promoting hair growth, and preventing or treating hair loss.

Specifically, when the concentration is lower than the concentration range described above, the effect of promoting hair growth, and preventing hair loss or treating hair may be insignificant or absent, whereas, whereas when the concentration is higher than the concentration range described above, the effect of promoting hair growth may also be insignificant.

In the composition of the present invention, the antioxidant refers to a material that is added for the purpose of terminating chain reactions of oxidation by acting on free radicals or peroxide generated during oxidation of active ingredients by oxygen in the air, and preventing progress of oxidation and deterioration of active ingredients.

In the present invention, the antioxidant may prevent deterioration of the effect of the composition including the substance P in promoting hair growth, and preventing or treating hair loss.

The antioxidant may be any conventional antioxidant that can be used in the art without limitation. Specifically, the antioxidant may be β-mercaptoethanol (β-ME), glutathione (GSH), ascorbic acid, vitamin E, beta-carotene, lycopene, coenzyme Q-10, selenium, chromium, magnesium, taurine, hypotaurine, trehalose, etc., but is not limited thereto. In the present invention, the antioxidant may be sodium thiosulfate.

The content of the antioxidant is not particularly limited as long as it can prevent the deterioration of the function of the composition in promoting hair growth, and preventing or treating hair loss, but may be 0.01% to 1% by weight based on the total weight of the composition of the present invention.

In the composition of the present invention, the surfactant refers to a material that maintains a homogenous liquid composition using a hydrophobic oil component.

The surfactant may be a general surfactant commonly used in the preparation of cosmetic compositions, such as anionic, cationic, non-ionic, or amphiphilic surfactants. Specifically, in the present invention, the surfactant may be polysorbate 80.

The content of the surfactant may be 0.001% to 0.1% by weight based on the total weight of the composition of the present invention, specifically, 0.006% to 0.1% by weight or 0.01% to 0.1% by weight.

In the composition of the present invention, the thickening agent refers to an additive that is added to provide viscosity. Specifically, the thickening agent of the present invention may be hydroxyethyl cellulose.

The content of the thickening agent may be 1% to 5% by weight based on the total weight of the composition of the present invention.

As used herein, the term “prevention” refers to all action taken to inhibit or delay hair loss by attenuating the regression phase and inducing the growth phase of hair follicles by the composition of the present invention.

As used herein, the term “treatment” refers to all action taken to inhibit or delay hair loss by attenuating the regression phase and inducing the growth phase of hair follicles by the composition of the present invention.

As used herein, the term “hair growth promotion” means an action taken to promote hair growth, which ultimately increases the proportion of hair in the growth phase in the entire hair. Accordingly, the term “hair growth promotion” inhibits hair loss caused by decreasing the proportion of hair follicles in the growth phase, and may have the same meaning as “hair loss improvement”, “hair loss prevention” and “hair loss treatment”.

As used herein, the term “hair loss alleviation” refers to an action taken to attenuate the regression phase of hair follicles and induce the growth phase thereof, thereby preventing hair loss. Accordingly, the term “hair loss alleviation” inhibits hair loss caused by decreasing the proportion of hair follicles in the growth phase, and may have the same meaning as “hair loss improvement”, “hair loss prevention” and “hair loss treatment”.

In the present invention, the pharmaceutical composition may be prepared as a medicine.

As used herein, the term “pharmaceutically acceptable carrier” refers to a carrier or diluent that does not cause irritation to an organism and does not abrogate activities and properties of the composition of the present invention in promoting hair growth, and preventing or treating hair loss. Examples of the pharmaceutically acceptable carrier used in the composition to be formulated into a liquid solution include saline, sterile water, Ringer's solution, buffered saline, an albumin injection solution, a dextrose solution, a maltodextrin solution, glycerol, ethanol, and a mixture of at least one component thereof, as those suitable for sterilization and in vivo use, and other conventional additive(s) such as an antioxidant, a buffer, a bacteriostatic agent, etc. may be further added as necessary.

Additionally, the pharmaceutically acceptable carrier of the present invention may include a non-naturally occurring carrier.

The pharmaceutical composition of the present invention is administered in a pharmaceutically effective dose. As used herein, the term “pharmaceutically effective dose” refers to an amount sufficient for the treatment or prevention of diseases at a reasonable benefit/risk ratio applicable to a medical treatment or prevention, and the level of the effective dose may be determined based on the severity of disease, drug activity, age, weight, health condition and sex of a patient, drug sensitivity, administration time, administration route and dissolution rate, and duration of treatment of the composition of the present invention, factors including drug(s) to be mixed or simultaneously used in combination with the composition of the present invention, and other factors well-known in the medical field. The pharmaceutical composition of the present invention may be administered alone or in combination with therapeutic agents for pterygium known in the art. It is important to administer an amount to obtain the maximum effect with a minimum amount without adverse effects considering the factors described above.

Still another aspect of the present invention provides a cosmetic composition, which includes substance P, an antioxidant, a surfactant, and a thickening agent, for promoting hair growth and alleviating hair loss.

As used herein, the terms “substance P”, “antioxidant”, “surfactant”, and “thickening agent” are as described above.

As used herein, the term “cosmetic composition” may be generally prepared as an emulsified formulation and a solubilized formulation. Examples of the emulsified formulation include nourishing cosmetic water, cream, essence, etc., and examples of the solubilized formulation include softening cosmetic water, etc. The cosmetic composition may be prepared in formulations selected from the group consisting of solution, suspension, emulsion, paste, gel, cream, lotion, powder, soap, surfactant-containing cleanser, oil, ampoule, power foundation, emulsion foundation, wax foundation, and spray, but is not limited thereto. Specifically, the cosmetic composition may be prepared in formulations of hypoallergenic cosmetic skin protective agent, softening cosmetic water, nourishing cosmetic water, nourishing cream, massage cream, essence, eye cream, serum, cleansing cream, cleansing foam, cleansing water, pack, cream, essence, spray or powder.

Additionally, the cosmetic composition of the present invention may further include at least one cosmetically acceptable carrier mixed to a general skin cosmetic composition. As conventional ingredients, for example, oil, water, surfactants, moisturizers, lower alcohols, thickening agents, chelating agents, colorings, preservatives, fragrances, etc. may be appropriately mixed, but are not limited thereto.

The cosmetically acceptable carrier contained in the cosmetic composition of the present invention may vary depending on the formulations.

When the formulation of the cosmetic composition is an ointment, paste, cream or gel, animal oil, vegetable oil, wax, paraffin, starch, tragacanth, cellulose derivatives, polyethylene glycol, silicone, bentonite, silica, talc, zinc oxide, or mixtures thereof may be used as a carrier ingredient.

When the formulation of the cosmetic composition is a powder or spray, lactose, talc, silica, aluminum hydroxide, calcium silicate, polyamide powder or mixtures thereof may be used as a carrier ingredient, and in particular, when it is a spray, a propellant such as chlorofluorohydrocarbon, propane/butane or dimethyl ether may be additionally included.

When the formulation of the cosmetic composition is a solution or emulsion, solvents, solubilizing agents or emulsifying agents may be used as a carrier ingredient, and for example, water, ethanol, isopropanol, ethyl carbonate, ethyl acetate, benzyl alcohol, benzyl benzoate, propylene glycol, 1,3-buthylglycol oil may be used. In particular, cottonseed oil, peanut oil, maize germ oil, olive oil, castor oil, sesame seed oil, glycerol aliphatic ester, polyethylene glycol or aliphatic ester of sorbitan may be used.

When the formulation of the cosmetic composition is a suspension, liquid diluents such as water, ethanol or propylene glycol, suspending agents, such as ethoxylated isostearyl alcohol, polyoxyethylene sorbitol ester and polyoxyethylene sorbitan ester, microcrystalline cellulose, aluminum metahydroxide, bentonite, agar, tragacant, etc. may be used as a carrier ingredient.

When the formulation of the cosmetic composition is a soap, alkali metal salts of fatty acids, fatty acid hemiester salts, fatty acid protein hydrolysates, isethionate, lanolin derivatives, aliphatic alcohols, vegetable oil, glycerol, sugars, etc. may be used as a carrier ingredient.

Still further another aspect of the present invention provides a health functional food, which includes substance P, an antioxidant, a surfactant, and a thickening agent, for promoting hair growth and alleviating hair loss.

The composition including substance P, an antioxidant, a surfactant and a thickening agent, hair growth promotion, and hair loss alleviation is as described above.

As used herein, the term “health functional food” refers to a food prepared or processed in the form of tablets, capsules, powders, granules, liquids, pills, etc. using raw materials or ingredients with useful functionality for the human body.

In particular, the term “functionality” refers to controlling nutrients for the structure of functions of the human body or providing useful effects for hygienic purposes, such as physiological effects, etc. The health functional food of the present invention may be prepared according to a method commonly employed in the art, and raw materials and ingredients commonly used in the art may be added when preparing the health functional food. Additionally, the formulation of the health functional food is not particularly limited so long as it is recognized as a health functional food.

The health functional food of the present invention may be prepared in various formulations, and the health functional food of the present invention uses a food as a raw material unlike generic drugs, and thus has no side effects that may occur during long-term administration thereof, is highly portable, and may be administered as an adjuvant for enhancing the effects of promoting hair growth and alleviating hair loss.

The health functional food of the present invention may further contain, as additional components, various flavoring agents or natural carbohydrates. The natural carbohydrates may include monosaccharides such as glucose, fructose, etc.; disaccharides such as maltose, sucrose, etc.; polysaccharides such as dextrin, cyclodextrin, etc.; and sugar alcohols such as xylitol, sorbitol, erythritol, etc. Natural sweetening agents such as thaumatin, a stevia extract, etc.; and synthetic sweetening agents such as saccharin, aspartame, etc. may be used as the sweetening agent.

In addition to the components described above, the health functional food of the present invention may contain various nutritional supplements, vitamins, electrolytes, flavoring agents, coloring agents, pectic acid and salts thereof, alginic acid and salts thereof, protective colloidal thickening agents, pH controlling agents, stabilizing agents, preservatives, glycerin, alcohols, carbonating agents used in carbonated drinks, etc. These components may be used alone or in combination. The ratio of such additives is not important, but is generally selected in a range of 0.000001 to 0.1 parts by weight, based on 100 parts by weight of the health functional food of the present invention, but is not particularly limited thereto.

Still another aspect of the present invention provides a method for promoting hair growth and alleviating hair loss, including administering a composition including substance P, an antioxidant, a surfactant, and a thickening agent to a subject in need thereof

In the method of the present invention for promoting hair growth and alleviating hair loss, the composition including substance P, an antioxidant, a surfactant and a thickening agent, promotion of hair growth and alleviation of hair loss is as described above.

The administration of the present invention may a method including application to the skin.

The composition of the present invention, which includes substance P, an antioxidant, a surfactant, and a thickening agent, may be administered to a subject in need thereof in an effective dose. The level of the effective dose can be determined by those skilled in the art based on the common knowledge to the extent that the desired effect of promoting hair growth and alleviating hair loss is exhibited.

### MODE FOR CARRYING OUT THE INVENTION

Hereinafter, the composition and effect of the present invention will be described in more detail by way of Examples. However, these Examples are given for illustrative purposes only, and the scope of the invention is not intended to be limited by these Examples.

### Example 1: Preparation of Composition, Which Includes Substance P, for Promoting Hair Growth and Alleviating Hair Loss

A novel formulation, in which sodium thiosulfate as an antioxidant, polysorbate-80 as a surfactant, and hydroxyethyl cellulose as a thickening agent were added to substance P, was prepared. Substance P was synthesized through a solid/solution phase using Fmoc-chemistry, a peptide synthesis technique, and purified through high performance liquid chromatography, and substance P having a purity of 85% or more was used.

TABLE 1
Composition Including Substance P

| Composition | Content |
|---|---|
| Substance P | 20 μg/mL |
| Sodium thiosulfate | 0.4% |
| Polysorbate 80 | 0.024% |
| Hydroxyethyl cellulose | 1.5% |

### Example 2: Confirmation of Effect of Composition Including Substance P on Proliferation of Dermal Papilla Cells

The effect of substance P on dermal papilla cell proliferation was analyzed by MTT assay. For the cell experiment, the contents of hydroxyethyl cellulose and polysorbate 80 in the composition including the substance P prepared as shown in Table 1 were used in a range that does not cause cytotoxicity.

More specifically, 1,000 μL of 0.5×10⁵ CFU/mL dermal papilla cell suspension was added to a 12-well culture plate and incubated for 48 hours. The medium (alpha-Minimum Essential Medium, alpha-MEM+10% FBS) was removed, and then 900 μL of a fresh medium (alpha-Minimum Essential Medium, alpha-MEM +5% FBS) was dispensed into each well, and then 100 μL of the composition including substance P diluted with phosphate buffered saline (PBS) was inoculated at concentrations of 30% and 50% (the substance P was contained at a concentration of 6 mg/mL and 10 mg/mL in the composition with concentrations of 30% and 50%, respectively). Additionally, substance P (30% and 50%) at the same concentrations as the composition including substance P was respectively mixed with phosphate buffered saline and treated.

Meanwhile, 100 μL of phosphate buffered saline was used as a negative control group. After inoculation, cells were incubated at 37° C. for 24 hours, and then the medium was removed. Then, the cells were washed with phosphate buffered saline three times. Subsequently, 900 μL of the medium was mixed with 100 μL of the MTT solution (0.5 mg/mL), and the mixed solution was treated to each well and allowed to react at 37° C. for 3 hours. Finally, the cells were washed with phosphate buffered saline and stirred in 150 μL of isopropyl alcohol solution for 3 hours, and absorbance at 570 nm was measured.

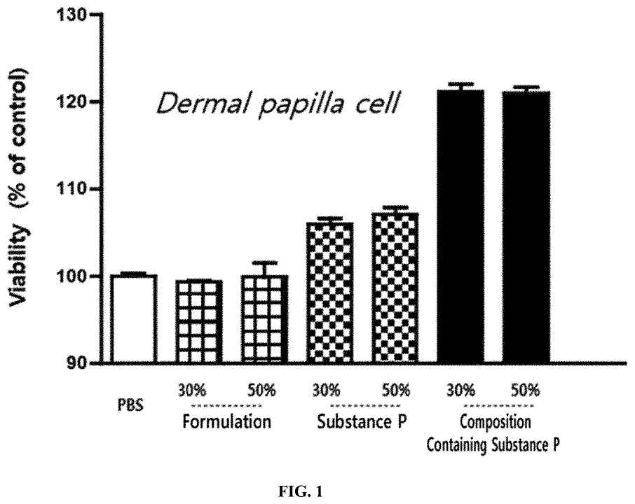

As a result, the composition including substance P showed an increase in the cell proliferation of dermal papilla cells in all concentration groups of 30% and 50%. Specifically, the cell proliferation was found to be 120% or more in all concentration groups (30% and 50%) of the composition including substance P, while the cell proliferation was found to be 107% in the group only treated with substance P. This indicates that the composition including substance P showed an effect on the proliferation of dermal papilla cells which is three times or higher compared to the group only treated with substance P (FIG. 1).

### Example 3: Chante of Alkaline Phosphatase (ALP) in Dermal Papilla Cells Treated by Composition Including Substance P

The change of alkaline phosphate (ALP) in dermal papilla cells by substance P was observed by reverse transcription polymerase chain reaction (RT-PCR).

More specifically, 0.27×10⁵ CFU/mL dermal papilla cells were incubated in a culture plate (100Φ dish) having an area of 55 cm² for 24 hours. After 24 hours, the medium (alpha-Minimum Essential Medium, alpha-MEM+10% FBS) was removed, and 9000 μL of a medium (alpha-Minimum Essential Medium, alpha-MEM) without fetal bovine serum was added, and then 1000 μL of the composition including substance P was inoculated at concentrations of 30% and 50%. Additionally, the group only treated with phosphate buffered saline was inoculated to the cells.

After 48 hours, dermal papilla cells were washed with phosphate buffered saline, and the cells were separated from the dish with 1× Trypsin-EDTA solution (0.05% trypsin, 0.53 mM Ethylenediaminetetraacetic acid (EDTA), Trypsin-EDTA, Welgene, Korea). Thereafter, RNA was isolated from the cell using the RNA extraction kit (TAKARA MiniBEST universal RNA extraction kit, TAKARA, Japan), and the extraction method is briefly described below. The cells were lysed with a cell lysis buffer, and RNA was isolated by spin column. After washing, the RNA was eluted, and the concentration was measured using a spectrophotometer (Ultrospec 6300 pro, Amersham Biosciences, USA).

In order to synthesize complementary DNA (cDNA), the cDNA synthesis kit (PrimeScript 1st strand cDNA synthesis kit, TAKARA, Japan) was used by adding RNA at the same concentration based on the measured concentration. Subsequently, ALP was quantitatively analyzed based on the synthesized cDNA by reverse transcription polymerase chain reaction.

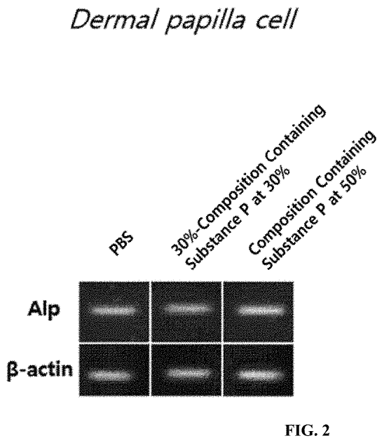

As a result, it was confirmed that the expression of alkaline phosphatase (ALP), a marker of dermal papilla cells in the growth phase, was not reduced in the dermal papilla cells proliferated by the composition including the substance P (30% and 50%) (FIG. 2).

From the result, it was confirmed that the composition including the substance P regenerated dermal papilla cells while maintaining the characteristics of the growth phase without altering the differentiation or characteristics of dermal papilla cells.

### Example 4: Confirmation of Effect of Composition Including Substance P on Proliferation of Germinal Matrix Cells

The effect of substance P on germinal matrix cell proliferation was analyzed by MTT assay. For the cell experiment, the contents of hydroxyethyl cellulose and polysorbate 80 in the composition including the substance P prepared were used in a range that does not cause cytotoxicity.

More specifically, 1,000 μL of 0.5×10⁵ CFU/mL germinal matrix cell suspension was added to a 12-well culture plate and incubated for 48 hours. The medium (alpha-Minimum Essential Medium, alpha-MEM+10% FBS+10 ng/ml bFGF) was removed, and then 900 μL of a fresh medium (alpha-Minimum Essential Medium, alpha-MEM+5% FBS+5 ng/ml bFGF) was dispensed into each well, and then 100 μL of the composition including substance P diluted with phosphate buffered saline (PBS) was inoculated at concentrations of 30% and 50%. Additionally, substance P (30% and 50%) at the same concentration as the composition including substance P was respectively mixed with phosphate buffered saline and treated. Meanwhile, 100 μL of phosphate buffered saline was used as a negative control group. After inoculation, cells were incubated at 37° C. for 24 hours, and then the medium was removed. Then, the cells were washed with the phosphate buffered saline three times. Subsequently, 900 μL of the medium was mixed with 100 μL of the MTT solution (0.5 mg/mL), and the mixed solution was treated to each well and allowed to react at 37° C. for 3 hours. Finally, the cells were washed with the phosphate buffered saline and stirred in 150 μL of isopropyl alcohol solution for 3 hours, and absorbance at 570 nm was measured.

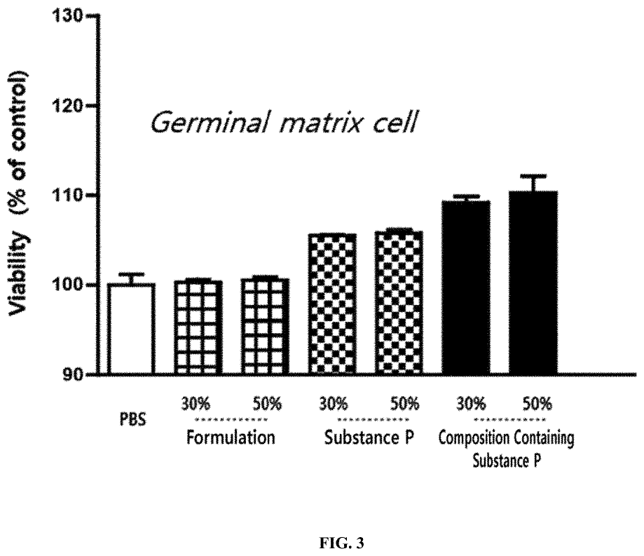

As a result, the cell proliferation in the group treated with substance P was found to be 105% at maximum, whereas the cell proliferation was increased to 110% or more in the concentration groups (30% and 50%) of the composition including the substance P (FIG. 3).

From the results, it was confirmed that the composition including the substance P showed an effect on the proliferation of germinal matrix cells which is about two times or higher compared to the group only treated with the substance P.

### Example 5: Confirmation of Hair Growth-Inducing Effect of Composition Including Substance P in Mice

The hair growth-inducing effect of the composition including the substance P was confirmed using mice.

More specifically, ketamine (Yuhan Co, Ltd, Korea) and rompun (Bayer Korea, Korea) were mixed to a final concentration of 24.2 mg/mL and 1.8 mg/mL, respectively, and then six-week-old female C3H/HeN mice (Orient Bio, Korea) were anesthetized with 80 μL of the anesthetic solution. After anesthesia, the hair on the back was removed using a clipper, and the remaining hair was completely removed using a hair removal cream. Then, eight mice with clean skin without wounds or remaining hair at the hair removal site were selected. Four mice were placed in each group so that mice with similar hair cycle were placed in each group, and thereafter the experiment was carried out. The phosphate buffered saline or the composition including the substance P at 50% concentration was intradermally injected in an amount of 50 μL each for a total of 200 μL at four sites on the back of the mice, at intervals of 3 to 4 days for 10 days from 2 days after the hair removal using an insulin syringe. The difference between C3H mice treated with the phosphate buffered saline or the composition including the substance P was analyzed using Welch's t-tests. Statistical significance between the groups was plotted on the graph as ***=P<0.001 and *=p<0.05. This statistic was analyzed based on Prism software version 5.

As a result, it was confirmed that the composition including the substance P at 50% concentration showed an increase in the area where the hair growth was promoted in the C3H mice compared to the group treated with phosphate buffered saline. Specifically, the group treated with the phosphate buffered saline had a resting state in the middle of the back of C3H mice, whereas the group treated with the composition including substance P at 50% concentration had a faster hair growth on both sides of the back and a rapid transition into the hair follicle growth phase even in the center (FIG. 4).

Additionally, the area of skin where the hair growth was induced was examined by the images taken at the same magnification by the Image J program.

As a result, it was confirmed that hair growth was induced in an average area of 3.5 cm² in the group treated with the phosphate buffered saline, whereas the area with the hair growth was twice larger in the group treated with the composition including the substance P with an average of 7.8 cm² (P<0.05) (FIG. 5).

Additionally, the length of the hair was randomly examined to demonstrate that the hair growth induction of substance P not only affected the area where hair grew but also increased the hair length.

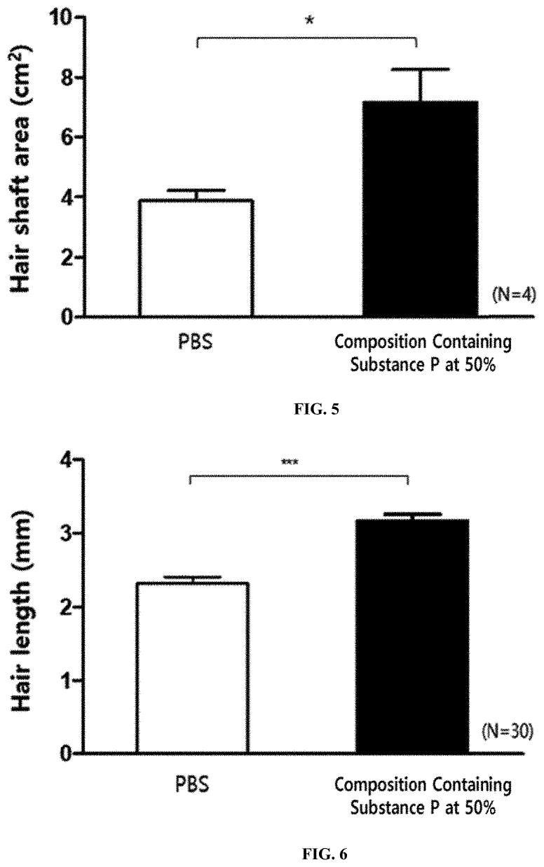

As a result, a total of 30 hairs was sampled and examined, and it was confirmed that the group treated with the phosphate buffered saline had an average hair length of 2.3 mm, while the group treated with the composition including the substance P at 50% concentration was found to have an average hair length of about 3.1 mm. The results obtained from the random sample examination demonstrated that the composition including substance P at 50% concentration had a hair growth effect of 135% relative to the group treated with the phosphate buffered saline, and these results were statistically significant (P<0.001) (FIG. 6).

From the results, it can be implied that the composition including the substance P at 50% concentration exhibited a hair growth effect through the simultaneous increase in the area of hair growth and the length of the hair.

### Example 6: Confirmation of Growth-Promoting Effect of Composition Including Substance P in Skin Follicles of Mice

The growth-promoting effect of the composition including the substance P in the skin follicles was confirmed using mice.

More specifically, after the procedure of Example 5, the skin tissue of the mice was collected in the direction of the hair follicle and in the direction vertical to the hair follicles, respectively, and fixed in a fixing solution (4% formaldehyde, Sigma, USA) for 24 hours. After 6 hours of washing to remove the fixing solution, the moisture that did not mix with a penetrant (paraffin, Leica, USA and Canada) was removed from the tissue through a dehydration process, and xylene (Daejung, Korea), which mixes well with the penetrant, was filled into the space in the tissue through a clearing process (dehydration process: 70% ethanol, Merck, Germany)->80% ethanol->90% ethanol->90% ethanol->95% ethanol->95% ethanol->100% ethanol->100% ethanol for 1 hour each, clearing process: xylene twice within 1 hour). Thereafter, an infiltration process was carried out to fill the penetrant into the tissue, followed by an embedding process to make a paraffin block. For hematoxylin (Sigma, USA) and eosin (Sigma, USA) staining, the paraffin block was cut into 8 μm and attached to slides (microscope slides, Marienfeld, Germany) coated with 2% 3-aminosilane, followed by drying in a 37° C. slide warmer for 12 hours. Thereafter, the slides were immersed in xylene 4 times for 7 minutes each through a deparaffinization process, and subjected to hydration for staining, and then washed with water. The slides were stained with hematoxylin for 1 minute for staining of cell nuclei and washed, and subsequently, the slides were stained with eosin for staining of cell cytoplasm and washed. Then, a dehydration process was carried out to prevent the contraction of the tissues and to maintain the same, and the tissues were protected by a cover glass using a mounting solution (Richard Allan Scientific, USA). The tissues were observed at 400X magnification under a microscope (BX41, Olympus, Japan) and photographed at the same magnification. The differences between the skin tissues treated with phosphate buffered saline and the composition including the substance P at 50% concentration were analyzed using Welch's t-tests. Statistical significance between the groups was plotted on the graph as ***=P<0.001. This statistic was analyzed based on Prism software version 5.

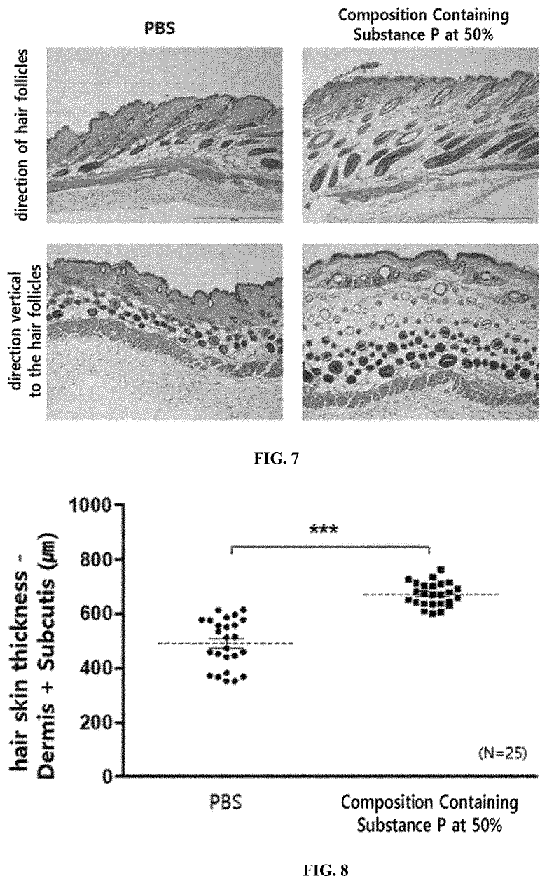

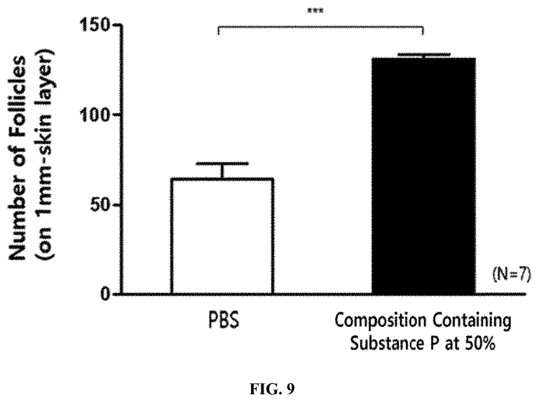

As a result, the appearance of the early stage of the growth phase, which is characterized by a thin subcutaneous layer, was observed in the group treated with the phosphate buffered saline for 12 days after hair removal, additionally, it was characterized by small number of hair follicles and size. However, the composition including the substance P at 50% concentration exhibited the skin tissue characteristics of the mature growth phase with a thick subcutaneous layer and a large number of hair follicles (FIG. 7).

Additionally, the skin thickness of 25 tissue images was analyzed for each group by Image J.

As a result, the group treated with the phosphate buffered saline showed an average thickness of the whole skin of 491 um, which was thinner than the group treated with the composition including the substance P at 50% concentration. The composition including substance P at 50% concentration showed a skin thickness of 671 um, indicating that the skin thickness was significantly increased to 137% relative to the group treated with phosphate buffered saline (P<0.001) (FIG. 8).

Further, the number of hair follicles in the skin tissue was analyzed by Image J.

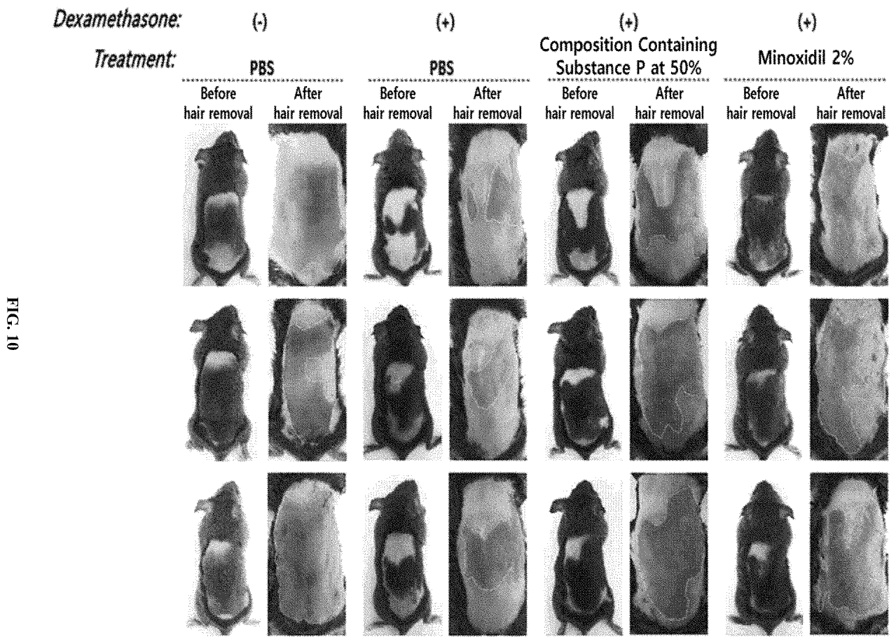

As a result, the group treated with phosphate buffered saline showed an average of 64 hair follicles in the skin of 1 mm in length, but 131 hair follicles were found in the group treated with the composition including the substance P at 50% concentration, confirming that the composition including the substance P at 50% concentration induced the proliferation of hair follicle by twice or more as compared to the phosphate buffered saline (P<0.001) (FIG. 9).

From the results, it can be implied that the composition including the substance P at 50% concentration showed a hair growth effect by inducing an increase in the thickness of the skin and the number of hair follicles.

### Example 7: Confirmation of Maintenance of Skin in Growth Phase of Composition Including Substance P in During Hair Regression Phase

The effect of the composition including the substance P on the maintenance of skin in the growth phase was confirmed using mice.

More specifically, ketamine (Yuhan Co, Ltd, Korea) and rompun (Bayer Korea, Korea) were mixed to a final concentration of 24.2 mg/mL and 1.8 mg/mL, respectively, and then six-week-old female C3H/HeN mice (Orient Bio, Korea) were anesthetized with 80 μL of the anesthetic solution. After anesthesia, the hair on the back was removed using a clipper, and the remaining hair was completely removed using a hair removal cream. Then, twelve mice with clean skin without wounds or remaining hair at the hair removal site were selected. Three mice were placed in each group so that mice with similar hair cycle were placed in each group, and thereafter the experiment was carried out. The phosphate buffered saline or the composition including the substance P at 50% concentration was intradermally injected in an amount of 50 μL each for a total of 200 μL at four sites on the back of the mice, at intervals of 3 to 4 days for 7 days from 6 days after the hair removal using an insulin syringe. Meanwhile, minoxidil was used as a positive control group, and 200 μL of minoxidil 2% (Pansidil Solution, Dongkuk Pharm., Korea) was allowed to be absorbed thoroughly after application to the back of the mice at the same period as the experiment above due to an inflammation reaction upon intradermal injection. 1 mL of dexamethasone (0.1% dexamethasone 21-acetate, Sigma, USA), which induces the regression phase, was applied to the back of the mice at intervals of 24 hours for 5 days from 10 days after hair removal and allowed to be absorbed thoroughly. 17 days after hair removal, the degree of hair growth of the mice and the area of maintenance of the growth phase after hair removal were photographed. One-way ANOVA was carried out to examine the difference between the entire groups, and the difference between individual means was analyzed using Tukey's test. Statistical significance between the groups was plotted on the graph as ***=p<0.001, **=p<0.01, *=p<0.05. This statistic was analyzed based on Prism software version 5.

As a result, the group treated with dexamethasone in combination with phosphate buffered saline showed a widely reduced area of black-skinned showing the characteristics of the growth phase compared to the mice untreated with dexamethasone. However, the group treated with the composition including the substance P at 50% concentration in combination with dexamethasone showed a wide area of black-skinned back through inhibition of the regression phase by dexamethasone (FIG. 10)

Additionally, the ratio of area of black skin relative to the area of whole skin with hair removal was confirmed for each group by the Image J program.

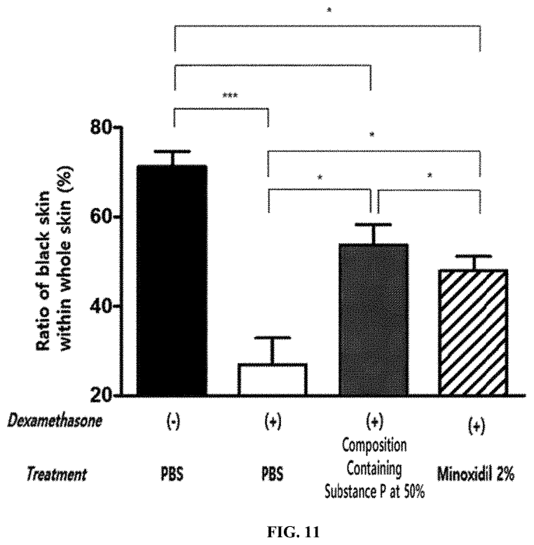

As a result, the group untreated with dexamethasone showed about 71% of black-skinned back relative to the total skin, but the group treated with dexamethasone in combination phosphate buffered saline maintained the black-skinned back by 30%, confirming that the regression phase was progressed, thereby showing a clear difference from the dexamethasone untreated group (Tukey's test, p<0.001). The groups treated with the composition including substance P at 50% concentration and minoxidil 2% maintained the black-skinned skin by 53% and 47%, respectively. This was believed that the composition including substance P at 50% concentration and minoxidil 2% inhibited the induction of the regression phase by dexamethasone by 56% and 46%, respectively (One-way ANOVA, F (3, 8)=17.03, p<0.001). Additionally, it was confirmed that the group treated with the composition including the substance P at 50% concentration had a similar or higher area of skin in the growth phase compared to the group treated with minoxidil 2% (FIG. 11).

From the results, it can be implied that the composition including the substance P showed a hair growth effect by inhibiting the induction of the regression phase of the skin and maintaining the skin in the growth phase.

### Example 8: Maintenance of Thickness and Shape of Skin in Growth Phase of Composition Including Substance P During Hair Regression Phase

The effect of the composition including substance P on the maintenance of thickness and shape of skin in the growth phase was confirmed using mice.

More specifically, after the procedure of Example 7, the skin tissue of the mice was collected in the direction of the hair follicles and fixed in a fixing solution (4% formaldehyde, Sigma, USA) for 24 hours. After 6 hours of washing to remove the fixing solution, the moisture that did not mix with a penetrant (paraffin, Leica, USA and Canada) was removed from the tissue through a dehydration process, and xylene (Daejung, Korea), which mixes well with the penetrant, was filled into the space in the tissue through a clearing process (dehydration process: 70% ethanol, Merck, Germany)->80% ethanol->90% ethanol->90% ethanol->95% ethanol->95% ethanol->100% ethanol->100% ethanol for 1 hour each, clearing process: xylene twice within 1 hour). Thereafter, an infiltration process was carried out to fill the penetrant into the tissue, followed by an embedding process to make a paraffin block. For hematoxylin (Sigma, USA) and eosin (Sigma, USA) staining, the paraffin block was cut into 8 μm and attached to slides (microscope slides, Marienfeld, Germany) coated with 2% 3-aminosilane, followed by drying in a 37° C. slide warmer for 12 hours. Thereafter, the slides were immersed in xylene four times for 7 minutes each through a deparaffinization process, and subjected to hydration for staining, and then washed with water. The slides were stained with hematoxylin for 1 minute for staining of cell nuclei and washed, and subsequently, the slides were stained with eosin for 10 seconds for staining of cell cytoplasm and washed. Thereafter, a dehydration process was carried out to prevent the contraction of the tissues and to maintain the same, and the tissues were protected by a cover glass using a mounting solution (Richard Allan Scientific, USA). The tissues were observed at 400× magnification under a microscope (BX41, Olympus, Japan) and photographed at the same magnification. One-way ANOVA was carried out to examine the difference between the entire groups, and the difference between individual means was analyzed using Tukey's test. Statistical significance between the groups was plotted on the graph as ***=p<0.001, **=p<0.01, *=p<0.05. This statistic was analyzed based on Prism software version 5.

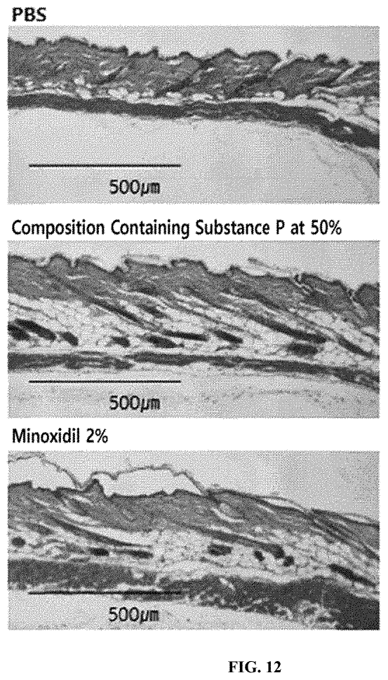

As a result, the group treated with dexamethasone in combination with phosphate buffered saline did not have a significant change in the thickness of the dermis, but it was observed that the thickness of the subcutaneous layer was rapidly reduced and the thickness of the skin as a whole was decreased significantly as the regression phase progressed. Additionally, the characteristics of the regression phase was maintained, while the location of hair roots remained in the dermis. In contrast, when the composition including the substance P at 50% concentration was treated, the subcutaneous layer was maintained and a large number of hair roots were located in the subcutaneous layer. In some cases, the characteristic of the regression phase (Type VI) that the dermal papilla cells were located far away from the hair was observed in the subcutaneous layer, but in most cases, the hair roots were located at the very bottom of the subcutaneous layer, while maintaining the appearance of the growth phase of the hair follicles. Such characteristics were not significantly different from the group treated with 2% minoxidil. In the case of 2% minoxidil, one of the characteristics of the regression phase that the degenerated dermal papilla cell bundles are crawled up was also observed (FIG. 12).

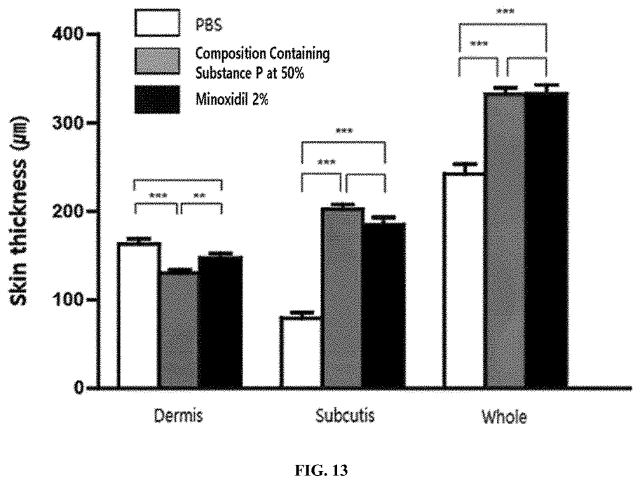

Additionally, the thickness of the dermis and the subcutaneous layers was confirmed by the Image J program. Skin thickness was measured by dividing the skin into the dermal layer (D), subcutaneous layer (SC), and whole layer (W), and 30 different areas were measured for each group and the results are shown in the graph of FIG. 13.

As a result, the thickness of the subcutaneous layer (SC) was reduced to an average thickness of 79 μm in the group treated with the phosphate buffered saline, whereas the composition including the substance P at 50% concentration showed an effect of maintaining the subcutaneous layer (SC) at an average of 202 μm. This result suggests that the composition including the substance P at 50% concentration inhibited the regression phase of the subcutaneous layer (SC) by about 255% compared to the phosphate buffered saline (Tukey's test, p <0.001). Additionally, it was confirmed that minoxidil had an effect of inhibiting the regression phase of the subcutaneous layer (SC) by 233% (FIG. 13).

From the results, it was confirmed that the composition including the substance P at 50% concentration and minoxidil 2% maintained the skin thickness of 130% or more compared the phosphate buffered saline (One-way ANOVA; F (2, 87)=30.65, p<0.001).

### Example 9: Maintenance of Number of Hair Follicles in Growth Phase of Composition Including Substance P During Hair Regression Phase

The effect of the composition including the substance P on the number of hair follicles in the growth phase was confirmed using mice.

More specifically, after the procedure of Example 7, the skin tissue of the mice was collected in the direction vertical to the hair follicles and fixed in a fixing solution (4% formaldehyde, Sigma, USA) for 24 hours. The hematoxylin and eosin staining was carried out in the same manner as in Example 8. The tissues were observed at 400× magnification under a microscope (BX41, Olympus, Japan).

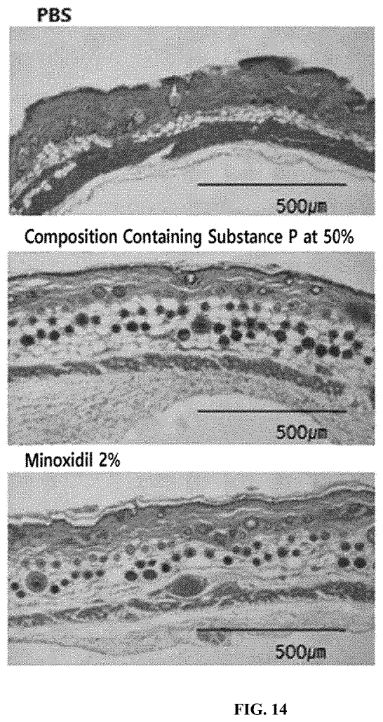

As a result, in the group treated with phosphate buffered saline in combination with dexamethasone, the number of hair follicles decreased from the dermis layer due to prolonged regression phase, and it was difficult to find hair follicles in the subcutaneous layer. In contrast, it was confirmed that in the group treated with the composition including the substance P at 50% concentration and the group treated with 2% minoxidil, many hair follicles were found in the dermis layer, and more hair follicles were found in the subcutaneous layer. One-way ANOVA was carried out to examine the difference between the entire groups, and the difference between individual means was analyzed using Tukey's test. Statistical significance between the groups was plotted on the graph as ***=p<0.001, **=p<0.01, *=p<0.05. This statistic was analyzed based on Prism software version 5 (FIG. 14).

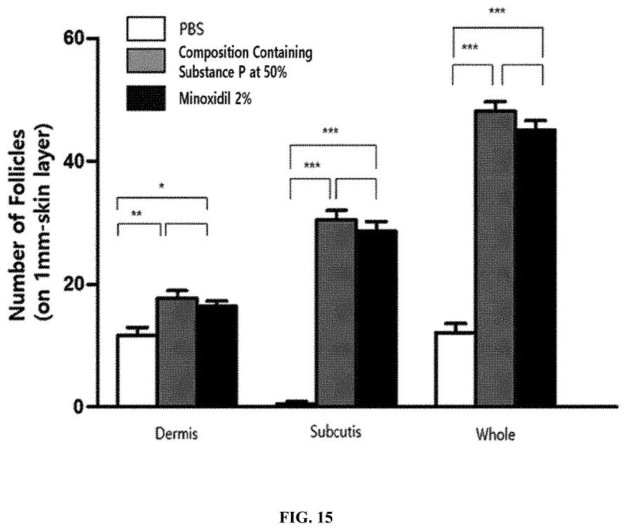

Additionally, the number of hair follicles in 1 mm of skin was measured for each group by the Image J program. The number of hair follicles were measured in 10 tissues per group by dividing the skin into D, SC, and W layers, and the results are shown in the graph of FIG. 15.

As a result, in the 1 mm of skin treated with dexamethasone in combination with phosphate buffered saline, an average of 11 hair follicles was found in the dermis layer and an average of about 12 hair follicles in the whole skin. However, an average of 18 and 48 hair follicles were identified in the group treated with the composition including the substance P at 50% concentration, confirming that the composition including the substance P at 50% concentration maintained the number of hair follicles by about 1.5 times in the dermis and by about 4 times in the whole skin compared to the phosphate buffered saline, respectively. In contrast, minoxidil 2% maintained the hair follicles by about 1.4 times in the dermis, about 56 times in the subcutaneous and about 3.7 times in the whole skin compared to the phosphate buffered saline, and thus the composition including the substance P maintained 7% more hair follicles found in the whole skin as compared to minoxidil (FIG. 15).

From the results above, it was confirmed that the treatment with the composition including the substance P at 50% concentration showed an excellent hair-promoting effect by maintaining more hair follicles compared to the treatment with minoxidil.

Those of ordinary skill in the art will recognize that the present invention may be embodied in other specific forms without departing from its spirit or essential characteristics. The described embodiments are to be considered in all respects only as illustrative and not restrictive. The scope of the present invention is therefore indicated by the appended claims rather than by the foregoing description. All changes which come within the meaning and range of equivalency of the claims are to be embraced within the scope of the present invention.

1. A pharmaceutical composition for promoting hair growth, and preventing or treating hair loss, comprising substance P consisting of an amino acid sequence of SEQ ID NO: 1, an antioxidant, a surfactant, and a thickening agent.

2. The pharmaceutical composition of claim 1, wherein the concentration of the substance P is 6 μg/mL to 10 μg/mL.

3. The pharmaceutical composition of claim 1, wherein the antioxidant is sodium thiosulfate.

4. The pharmaceutical composition of claim 1, wherein the surfactant is polysorbate 80.

5. The pharmaceutical composition of claim 1, wherein the thickening agent is hydroxyethyl cellulose.

6. A cosmetic composition for promoting hair growth and alleviating hair loss, comprising substance P consisting of an amino acid sequence of SEQ ID NO: 1, an antioxidant, a surfactant, and a thickening agent.


SEQUENCE LISTING (1 sequence)

SEQ ID NO: 1 - protein, 11 aa, Artificial Sequence
  Substance P
RPKPQQFFGLM